(19) United States
(12) Patent Application Publication
(10) Pub. No.: US 2002/0141598 A1
(43) Pub. Date: Oct. 3, 2002

(54) Arrangement for activating and deactivating automatic noise cancellation (ANC) in a mobile station

(75) Inventor: Pekka Vayrynen, Oulu (FI)

Correspondence Address: Michael C. Stuart, Esq., Cohen, Pontani, Lieberman & Pavane, Suite 1210, 551 Fifth Avenue, New York, NY, 10176, US

(73) Assignee: Nokia Corporation

(21) Appl. No.: 10/096,988

(22) Filed: Mar. 13, 2002

(30) Foreign Application Priority Data
Mar. 29, 2001 (FI) 20010654

(51) Int. Cl. A61F 11/06; G10K 11/16; H03B 29/00

(52) U.S. Cl. 381/71.1; 381/57; 381/94.5

(57) ABSTRACT

An arrangement for activating and deactivating automatic noise cancellation (ANC) in a mobile station, in which there are an ANC facility (3) for suppressing background noise in an incoming audio signal, as well as circuits for activating and deactivating the ANC, includes automatic detection (10) of the need for ANC according to a selected criterion, for example, a set noise level, the detection circuit configuration (10) being arranged to automatically control the activation and deactivation of the ANC facility (3).

### FIELD OF THE INVENTION

The present invention relates to an arrangement for activating and deactivating automatic noise cancellation (ANC) in a mobile station, in which there is an ANC circuit for removing background noise from an audio signal and circuits for activating and deactivating ANC.

### BACKGROUND OF THE INVENTION

Automatic noise cancellation is used to remove background noise from an incoming audio signal. This feature is generally separately activated and deactivated. Its continuous use is not desirable, as it increases the power consumption of the mobile station. Usually the user must select, from the mobile station's settings, the method of using the ANC, which can be, for example, ‘Always ON’ or ‘Manual’. The former results in unnecessary power consumption while the latter is difficult for the user, because it demands several key strokes, which additionally must often be made during a call.

The theory and operating methods of automatic noise cancellation are widely known. U.S. Pat. Nos. 5,406,635 (Järvinen) and 5,839,101 (Vähätalo) disclose certain methods for limiting background noise in a mobile station. The publication ‘IEEE Signal Processing Magazine’; S. J. Elliot, August 1993 features an extensive presentation of the history of noise cancellation technology and certain present methods. Background noise cancellation can be based either on the analysis of frequency fields (e.g., adaptive filters) or on active feedback on background noise obtained with the aid of an additional microphone, in cases in which this is possible.

All the methods have the drawback that they cause an increase in power consumption, which, in mobile stations, means a shorter period of operation for each battery charge. Thus, the use of ANC is only desirable when it is really necessary.

The present invention is intended to create an arrangement for activating and deactivating automatic noise cancellation when it is required.

### SUMMARY OF THE INVENTION

According to the invention, this purpose is achieved in such a way that the system includes an automatic circuit 42 configuration detecting a need for ANC, according to a selected criterion, the detection circuit configuration being arranged to automatically control the activation and deactivation of ANC. According to one preferred embodiment, the detection circuit configuration includes a comparison circuit for comparing the set criterion value with the volume level of the mobile station device, in such a way that the comparison circuit activates ANC at the set sound volume value.

According to a second preferred embodiment, if there is an additional microphone in the mobile station for measuring background noise, the detection circuit configuration includes a comparison circuit for comparing the set criterion value with the measured value of the background noise, in such a way that the comparison circuit activates ANC at the set volume level of the background noise.

Once the ANC setting criteria has been made, the user need no longer take care of activating and deactivating ANC.

With the aid of the invention, automatic noise cancellation will only be activated when it is really necessary, thus avoiding unnecessary power consumption. Other advantages and embodiments of the invention will become apparent in connection with the examples described later.

Other objects and features of the present invention will become apparent from the following detailed description considered in conjunction with the accompanying drawings. It is to be understood, however, that the drawings are intended solely for purposes of illustration and not as a definition of the limits of the invention, for which reference should be made to the appended claims.

### BRIEF DESCRIPTION OF THE DRAWINGS

In the following, the invention is examined with reference to the accompanying figures, in which:

FIG. 1 shows a schematic diagram of a mobile station equipped with noise cancellation,

FIG. 2 shows a flow chart of the noise cancellation of the embodiment of FIG. 1,

FIG. 3 shows a schematic diagram of a mobile station according to a second embodiment, and

FIG. 4 shows a flow chart of the noise cancellation of the embodiment of FIG. 3.

### DETAILED DESCRIPTION OF THE INVENTION

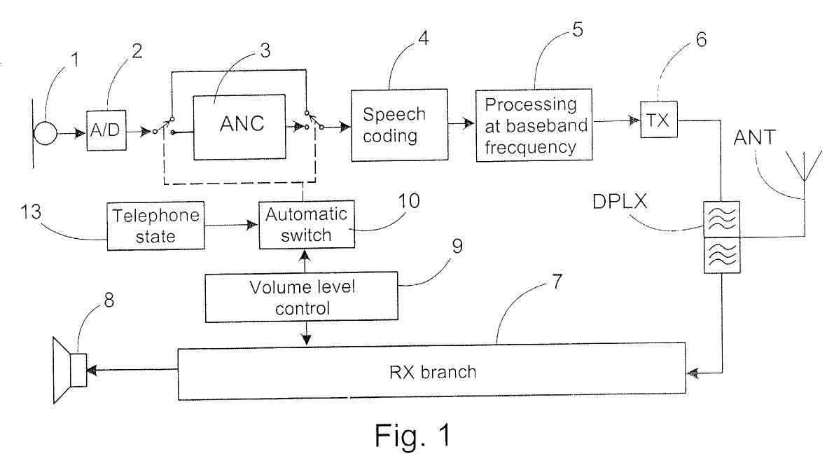

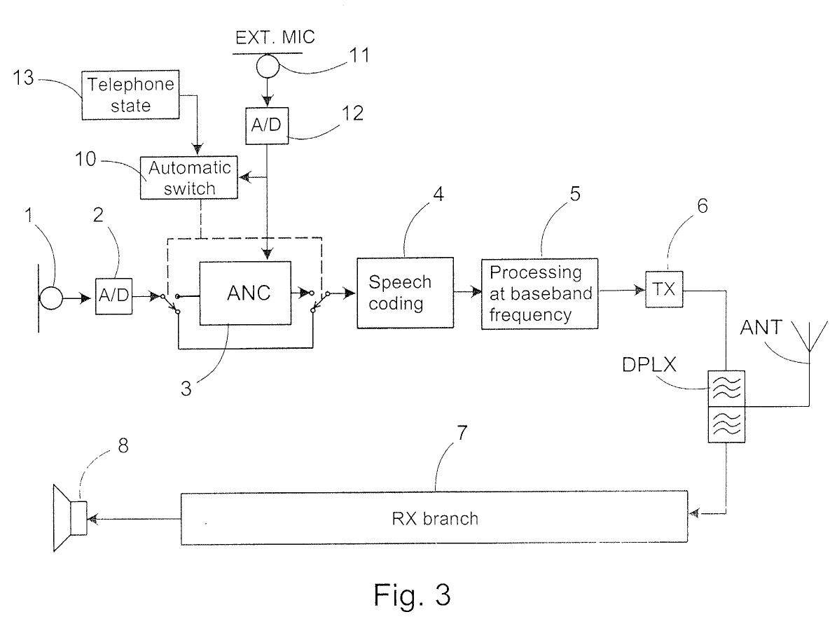

In both cases, the schematic diagrams of the examples of FIG. 1 and 3 show the operational configuration of the mobile station. Naturally, the signal is processed digitally. Thus, the ‘bypass connection’ of the noise cancellation (ANC) takes place by forwarding the flow of bits, which arrives at the ANC's incoming register, directly to the following stages, i.e. to the input register of the speech coding, without processing. When the noise cancellation has been activated, before the flow of bits arriving at the incoming register of the ANC is forwarded, it is subject to processing, which is, as such, known.

The invention can be implemented in different types of mobile station (TDMA, CDMA, digital/analog).

The schematic diagram according to FIG. 1 shows a digital mobile station, in which the speech to be sent, which comes to the microphone 1, is converted into digital form by an A/D converter 2, the noise is suppressed, if necessary, by noise cancellation (ANC facility 3), and the speech is coded in speech coding 4, after which processing of the baseband frequency signal takes place in the block 5, as is known. Next, the signal is converted to a radio frequency and transmitted 6 through a duplex filter (DPLX) and an antenna (ANT). The known operations of the receiver branch 7 are carried out when speech is received in the receiver, and it is reproduced in the loudspeaker 8. In addition, FIG. 1 is marked with a means 13, the significance of which is explained later, for showing the state information of the mobile station.

In the description according to FIG. 1, volume level control 9, the information available from which is exploited by the automatic detection circuit configuration 10 to detect a need for noise cancellation, is connected to the receiver branch 7. This too is actually implemented by means of software, which is explained later. In any event, the noise cancellation is activated and deactivated automatically, with only the receiver volume level control value and the state information of the mobile station being exploited to detect a need for it.

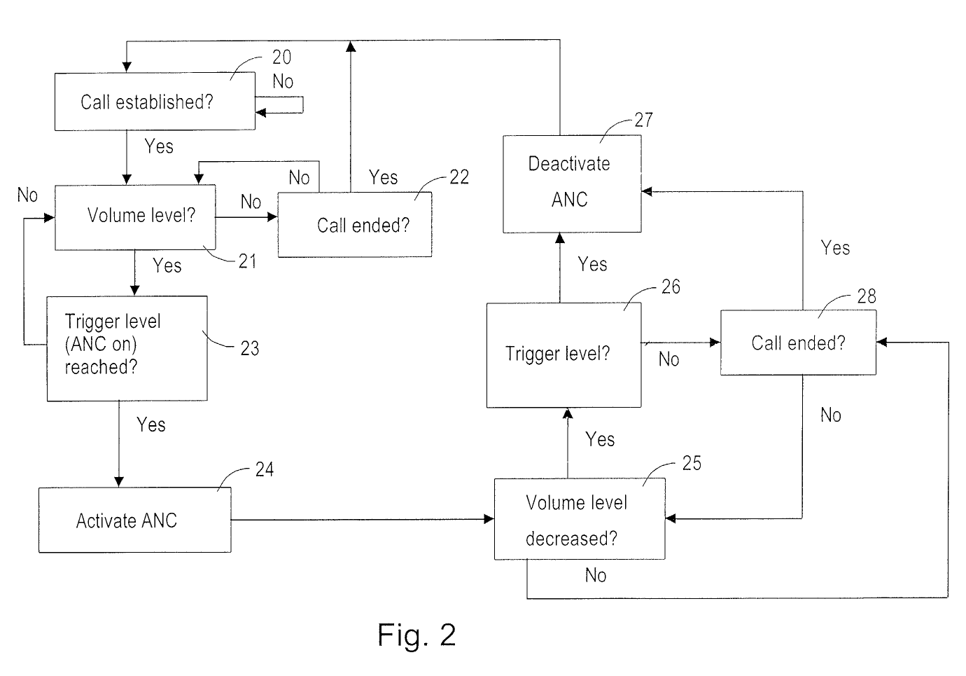

Automatic detection can be implemented by means of software in many different ways. Thus, FIG. 2 shows only one possible implementation, in the form of a program flow chart. When the mobile station is closed, the program monitors the loop formed by an initial check 20. Once a call is connected, the execution changes to monitoring the level of the volume level of the loop 21. If the volume level is not increased, the program returns to check 22 the status. If the volume level is increased, the execution moves to checking 23 the trigger level and returns, unless the trigger level is reached. If the check 23 detects that the trigger level has been reached, the program activates the ANC, stage 24. From this, the execution must move to the checking stages in the opposite direction. When a call is connected and remains at the same sound level, the execution runs around the loop in stages 25, ‘Volume level reduced’ and 28, ‘Call ended?’. If the volume level is reduced, check 26, ‘Trigger level achieved’, follows, from which the execution returns to stage 25, if the trigger level has not been achieved and the call is still connected. If the trigger level has been reached, the program deactivates the ANC in stage 27 and returns to the initial check 20.

The program flow can also incorporate more complex initializations, for example, an initial state check of the volume level setting and, according to it, a move to either stage 21 or stage 25.

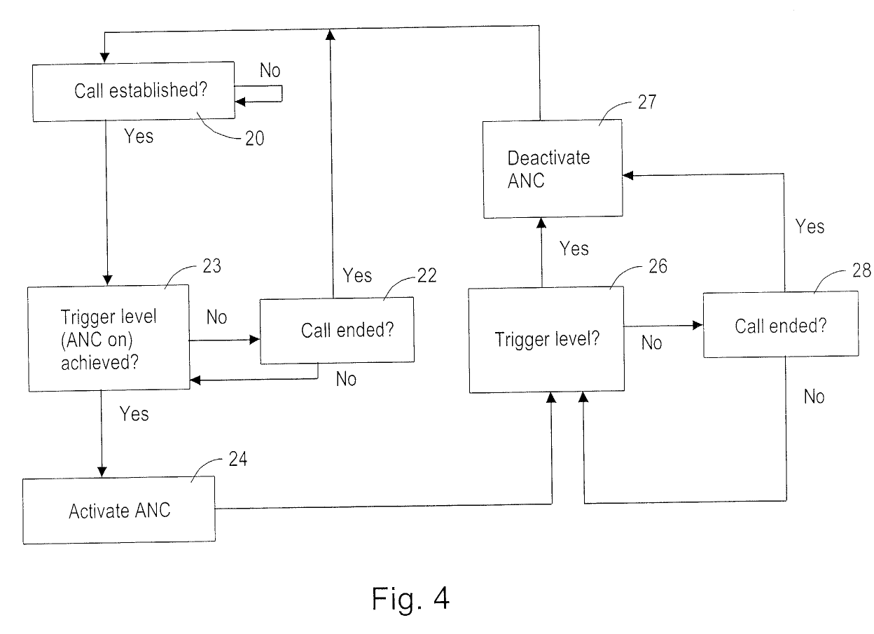

FIG. 3 shows a schematic diagram of a second embodiment while FIG. 4 shows a corresponding flow chart. The same reference numbers as above are used for operationally corresponding components. Thus, in the digital mobile station according to FIG. 3, the speech that comes from the microphone 1 is also converted using an A/D converter 2, the noise is cancelled, if necessary, by noise cancellation (ANC facility 3), and the speech is coded in speech coding 4, after the baseband frequency signal is processed in block 5. Similarly, after this the signal is converted to a radio frequency and transmitted by a transmitter 6, through a duplex filter (DPLX) and an antenna (ANT). The known operations of the receiver branch 7 are carried out in this case too, when speech is received and it is repeated by the loudspeaker 8.

Unlike in FIG. 1, in the embodiment of FIG. 3, a separate microphone 11 and an A/D converter 12 are used to detect background noise. This is primarily exploited in the noise cancellation itself (ANC facility 3), but also in detecting 10 a need for ANC. Here too, detection 13 of the state of the mobile station is exploited when detecting a need for ANC. The corresponding program flow chart is somewhat simpler, because the criterion value required for the program is constant, FIG. 4. Thus, from the initial state check 20 the flow goes to monitor the first trigger level check 23, from which, if the trigger level is exceeded, ANC is activated, stage 3, and the program then goes to the second trigger level check 26. From here the execution returns to the initial check 20, through the deactivation of the ANC, stage 27, if the trigger level has been reached (downwards). Otherwise, the execution runs around a loop through the call state check 28 and the second trigger level check 26.

In this case too, the program can be supplemented and adapted in many different ways.

Thus, while there have been shown and described and pointed out fundamental novel features of the present invention as applied to a preferred embodiment thereof, it will be understood that various omissions and substitutions and changes in the form and details of the devices described and illustrated, and in their operation, and of the methods described may be made by those skilled in the art without departing from the spirit of the present invention. For example, it is expressly intended that all combinations of those elements and/or method steps which perform substantially the same function in substantially the same way to achieve the same results are within the scope of the invention. Substitutions of elements from one described embodiment to another are also fully intended and contemplated. It is the intention, therefore, to be limited only as indicated by the scope of the claims appended hereto.

What is claimed is:

1. An arrangement for activating and deactivating automatic noise cancellation (ANC) in a mobile station, in which there is an ANC facility for cancelling background noise of an incoming audio signal and circuits for activating and deactivating ANC, characterized in that the arrangement includes an automatic circuit configuration for detecting a need for ANC, according to a selected criterion, which detection circuit configuration is arranged to automatically control the activation and deactivation of the ANC facility.

2. A arrangement according to claim 1 in a mobile station equipped with sound level control, characterized in that the detection circuit configuration includes a comparison circuit for comparing the set criterion value with the volume level control value of the mobile station device, in such a way that the comparison circuit activates the ANC facility at the set volume level value.

3. An arrangement according to claim 2 in a mobile station, which is equipped with a digital operating system controlling its functions, characterized in that the arrangement includes stages carried out by means of software:
a) a first comparison of the set value of the volume level with a criterion value, if the set value is raised,
b) return to the first comparison, if the criterion value is not exceeded,
c) activation of the ANC facility, if the criterion value is reached, and a move to a second comparison (d-f),
d) a second comparison of the set value of the volume level, if the set value is reduced,
e) return to the second comparison, if the criterion value is not gone below,
f) deactivation of the ANC facility, if the criterion value is gone below, and a move back to the first comparison (a-c).

4. An arrangement according to claim 3, characterized in that the arrangement also includes one or several checks to examine the operating state, so that, in a non-active state:
if the ANC is activated, it is deactivated,
a move is made from the first and/or the second comparison to a comparison of the operating state, from which a return is made to the first comparison until the operating state is activated.

5. An arrangement according to claim 1 in a mobile station, in which there is an additional microphone for detecting background noise, characterized in that the detection circuit configuration includes a comparison circuit for comparing the set criterion value with the value of the measured background noise, in such a way that the comparison circuit activates the ANC facility at a set value of the volume of the background noise.

6. An arrangement according to claim 5 in a mobile station, which is equipped with a digital operating system controlling functions, characterized in that the arrangement includes stages carried out by means of software:
a) a first comparison of the background noise with the criterion value,
b) a return to the first comparison, if the criterion value is not exceeded,
c) activation of the ANC facility, if the criterion value is reached, and a move to a second comparison (d-f),
d) a second comparison of the background noise with the criterion value,
e) a return to the second comparison, if the criterion is not gone below,
f) deactivation of the ANC facility, if the criterion value is gone below, and a move back to the first comparison (a-c).

7. An arrangement according to claim 6, characterized in that the arrangement also includes one or several checks to examine the operating state, so that in a non-active state:
if the ANC facility is activated, it is deactivated, and
a move is made from the first or second comparison to a comparison of the operating state, from which a return is made to the first comparison, until the operating state is activated.
